(19) United States
(12) Patent Application Publication
(10) Pub. No.: US 2009/0068673 A1
(43) Pub. Date: Mar. 12, 2009

(54) UTILIZATION OF COMPATIBLE SOLUTES TO IMPROVE THE PERFORMANCE OF THE TECHNIQUES USING IMMOBILIZED BIOLOGIC MATERIALS

(75) Inventors: Jorge Miguel Pereira De Oliveira Da Silva Santos, Oeiras (PT); Catia Machado, Oeiras (PT); Ana Catarina Varela Raposo, Oeiras (PT); Stefano Volinia, Ferrara (IT); Nicoletta Mascellani, Ferrara (IT); Steven John Setford, Bedfordshire (GB); Elisabeth Loose, Sceaux (FR); Christopher David Maycock, Oeiras (PT); Maria Rita Mendes Bordalo Ventura Centeno Lima, Oeiras (PT); Pedro Lamosa Antonio, Oeiras (PT); Isabel Sequeira Lopes, Oeiras (PT); Helena Santos, Oeiras (PT); Alcino Orfeu De Leao E Flores, Charneca da Caparica (PT)

Correspondence Address: SUGHRUE MION, PLLC, 2100 PENNSYLVANIA AVENUE, N.W., SUITE 800, WASHINGTON, DC, 20037, US

(73) Assignees: STAB VIDA, Investigacao e Servicos em Ciencias Biologicas, Lda, Oerias (PL); BITOP AG, Witten (DE)

(21) Appl. No.: 12/280,503

(22) PCT Filed: Feb. 23, 2007
(86) PCT No.: PCT/PT07/00013

§ 371 Date: Aug. 22, 2008

(30) Foreign Application Priority Data
Feb. 24, 2006 (PT) 103441

(51) Int. Cl. C12Q 1/68; C12Q 1/02; C12Q 1/00; G01N 33/00

(52) U.S. Cl. 435/6; 435/29; 435/4; 436/94; 436/86

(57) ABSTRACT

This invention is based upon the application of compatible solutes to immobilized biological materials or to immobilize in order to improve the technical performance using the same, namely nucleic acid and proteins or biosensors micronets. The above mentioned compatible solutes are selected from a group that includes Mannosylglycerate (MG) and its 3 derivatives manno-sylglyceramide (MGA), -mannosylglycerol (MGOH), and mannosyl-lactate (MGLac), 1,1-di-glycerol phosphate (DGP), ectoin, hydroxyectoin, and or derivatives or combinations thereof. The improved performance of the above mentioned techniques might be due to: a better state of preservation of the material to be immobilized, increasing the life expectancy of the material, and/or decreasing the storage and/or transport requirements, a more efficient and uniform deposition of the material on the support, more efficiency and/or specificity of hybridation of the marked material, all this originating an increase of the signal-to-noise ratio.

### TECHNICAL DOMAIN

This invention is based on the application of compatible solutes with the biomaterials stabilizing ability, as well as, its derivates and combinations, in order to improve the technical performance using immobilized biologic materials, whether in the development of the utilization of perfected methodologies, whether in the conservation and support transport and samples to be utilized in those same techniques. These techniques comprise the arising from the DNA micronets, already implemented, as well as, the related to the protein micronets and the developing bio-sensors. The positive effects of the compatible solutes in the referred applications are based on the stabilization/protection effect, which these compounds present in what regards the in vitro nucleic acids, and the already acknowledged similar effect that the compatible solutes show in regards to the proteins.

### PREVIOUS STATE OF THE ART

A great variety of microorganisms accumulate within the solute cells of low molecular weight in high levels, when subject to extreme environmental conditions, namely, osmotic or thermal aggressions. These solutes are generally employed, as osmolytes in halotolerant or halophilic organisms. The variety of accumulated solutes is relatively low, and circumscribes to few compound categories. These include polyols, sugars and derivatives, amino acids and substances that derive from amino acids, betains, ectoins, and oligopeptids. (Brown, 1979).

Apart from being compatible with the normal cellular activity, it is thought that osmolytes, when accumulated in environmentally aggressive conditions, have a stabilizing effect over the cellular compounds. It is established that the compatible solutes have the protein stabilizing effect, whether when they are subject to thermal aggression, whether in the presence of denaturing agents, such as, detergents, organic solvents, urea or guanidine chloride. (Arakawa and Timasheff, 1985).

Mannosylglycerate (MG) is a compatible mannose derived solute, which is accumulated by some thermophiles and hyperthermophiles organisms, mainly as a response to the osmotic aggression. (Santos e da Costa, 2002). The MG was initially identified in red algae, of the Ceramiales family (Bouveng et al., 1955), never been observed in mesophile prokaryotes. Among the microorganisms that accumulate MG, the ones that stand out are the thermophiles and hyperthemophiles, moderately halophiles of the Archaea and bacteria domains, namely among the genus Pyrococcus, Thermococcus, Archaeoglobus, Aeropyrum, Thermus and Rhodotermus (Martins e Santos, 1995; Lamosa et Al., 1998; Nunes et al., 1995).

Based on the observation of the invariable association of the mannosylglycerate to organisms characteristic from high temperature environments, it was speculated that this compound would work as biologic structure protector in a higher manner, in comparison to its mesophilic homologous, against high temperature effects. In fact, subsequent experiences show that the MG, as well as, its amidic derivative the manno-sylglyceramide (MGA), are doubtlessly excellent protein stabilizers against thermal denaturation of proteins (Ramos et al., 1997; Santos et al., 1998). The same high stabilising ability was also shown to some synthetic variants of mannosylglycerate, particularly to mannosylglycerol and to mannosyl-lactate (submitted patent application).

The glycerol phosphate is a rare compatible solute, only found in species of the Archaeoglogus genus, a hyperthemophiles archaeon. Once investigated, this compound also revealed to have remarkable properties for the stabilisation of proteins in solution.

The ectoins (both the simple ectoin as well as its hydroxilated version, the hydroxyectoin) occur in halophilic and mesophilic organisms. For these compounds, protective properties are descried which include not only the protein, nucleic acids, and vaccines structural protection in solution, as well as suppression properties of oxygen free radicals and tissue hydration, among others (Knapp et al., 1999; Malim et al., 1999).

The mechanism through which, these solutes of high stabilizing capability of biologic materials in solution work, is yet to be clarified. One of the most commonly spread out theories is based on the fact that, an aqueous solution of low molecular weight compounds presents physical properties other than water; particularly, being observed an increase of the superficial tension of the liquid. This increase has as consequence a greater state of structural organization of the waters, in the hydration layers of the protein. This effect leads to a decrease of the entropy in the hydration layers, process which is per se thermodynamically unfavourable, therefore leading to a minimization of the interface area protein/water. The protein denaturation process implies a lateral chain protein overture and an increase of the exposed area to the solvent. As such, the addition of solutes to a protein solution decreases the exposed area of the protein, posing difficulties to the denaturation process. The outcome is the higher native protein ratio/denatured protein. (Arakawa and Timasheff 1985).

In what regards the nucleic acids, there are no previous results that show the effect of the compatible solutes in its in vitro stability. It is known that the double DNA helix depends on the size of the molecule; the longer it is, the higher the fusion temperature will be, or melting (Tₘ). The guanine-cytosine pairing content (% GC) also influences the Tₘ, in ratio: Tₘ=81.5+0.41 (% GC)° C. (Breslauer et al., 1986; Freier et al 1986), a constant concentration of sodium chloride (NaCl) of 1M. A saline concentration (e.g. [KC]), is important because the salt, due to its polarity, it partially annuls the negative charges present in the DNA phosphate groups, which have the tendency to repel each other. This DNA molecule stabilizing effect, due to the ionic force of the salt present in the solution, it is mathematically translated by the expression: Tₘ=Tₘ std+16.6 (log C), where C<1M and C=saline concentration, e.g. KCl. The natural implication of this mathematical expression is that the DNA molecule stabilization; as big as the saline concentration: an ionic strength effect named polyelectrolyte (Record et al., 1978; Manning, 1978; Le Bret and Zimm, 1984). Lastly, the number of erroneous pairing or mismatches influences the Tₘ: Tₘ=Tₘ std−0.71 (% mismatch). The larger the number of imperfect matches (other than the natural ones, Watson and Crick, A-T and G-C), the less stable the inter-chain connection, therefore, lower the Tm (Breslauer et al., 1986; Freier et al 1986).

It can be concluded that the two factors that affect the Tₘ are, the polyelectrolyte effect (Record et al., 1978; Manning, 1978; Le Bret and Zinun, 1984) and the composition of the DNA molecule in terms of % GC (Breslauer et al., 1986; Freier et al 1986). Thus the Tₘ dependency on the saline concentration and % GC it can be empirically represented by the expression: Tₘ=16.6 log(S)+41.5 (X_{GC})+81.5, where Tₘ=denaturation temperature, S=total concentration of monovalent ions (mol/L), and X_{GC}=molar fraction of GC base pairs in DNA. From this expression, it can be concluded that the polyelectrolitic effect, is strongly affected by the ionic force of the solvent, whereas the % GC effect is influenced by any additive that alters the hydrofobicity of the solvent (Rees et al, 1993).

These new observations open a window of opportunity for the application of compatible solutes of high stabilizing capability to techniques, by using immobilized biologic materials, particularly, micronets and biosensors. It is also important to mention that the beneficial effect of these new applications does not only strain with the stabilizing/structural effects, conferred by these solutes to the immobilized biological materials or to be immobilized, but also, and in a fundamental way, with and added specificity that these can confer to the referred techniques, as it will become clear whilst reading the following examples (see example section).

These new observations open a window of opportunity for the application of compatible solutes of high stabilizing capability to techniques, by using immobilized biologic materials, particularly, micronets and biosensors. It is also important to mention that the beneficial effect of these new applications does not only strain with the stabilizing/structural effects, conferred by these solutes to the immobilized biological materials or to be immobilized, but also, and in a fundamental way, with and added specificity that these can confer to the referred techniques, as it will become clear whilst reading the following examples (see example section).

The DNA, RNA and protein micronet technology is now-a-days, one of the most important supports in the molecular biology field, in what regards the analysis of the global expression of the transcriptomes and proteomes and is based on the deposition and immobilization of the biologic material (DNA, RNA, and protein) in sites (positions) with predefined coordinates on a solid surface. The basic principle behind the operating networks is but a specific interaction between the immobilized molecules on the solid support (probes) and the molecules that interact specifically with them (Targets). A hybridation solution containing chemically marked target molecules, promotes the contacts between these and the immobilized probe molecules. After the hybridation step, the micronet is washed in order to remove non-interactive target molecules and non-incorporated marking agents. Finally, the marking which comes from the interactive target molecules with the probe molecules (e.g. nucleic acid duplexes DNA/DNA, DNA/RNA, RNA/RNA or protein/protein) is measured and quantified through a detector coupled to appropriate software and hardware. Protein micro networks case can be divided into two ranks: those of direct phase which, just like the nucleic acids micronets, analytes, or target molecules are captured from the hybridation solution and those of the reverse phase wherein the analytes are fixed on the solid support. Typically, in the direct phase micronet proteins, a probe molecule; an antibody is immobilized in a solid support in order to capture the analytes, that call be purified proteins or in complex solutions as; cellular lysates or cellular tissue preparations. The analytes that interconnect to the immobilized antibodies are detected through a direct marking or through a secondary marked antibody. In the case of the reverse phase micronet proteins, the analytes (typically purified proteins or cellular lysates) are fixed directly into a solid support and antibodies or interactive proteins (whole proteins, connection domains or simply peptides) are applied to a hybridation solution. In this case, the analytes are directly marked or detected through techniques which involve signal amplification.

Despite the enormous variety of already available micronets, the networks containing DNA immobilized oligonucleotides in a solid surface chemically pre-treated, are by far the most used in all fields of investigation and diagnosis. Namely, GeneChip® micronets type from Affymetrix, are at the moment the more used at a global scale for diagnose testing and comparative genetic expression, allowing a global genetic expression analysis of several organisms. The GeneChip® network type from Affymatrix, despite it enormous utilization, are in a recent developing phase having still the possibility to improve its performance in several parameters. These parameters, often, make it difficult to compare the obtained experiment results and the different platforms, seriously affecting its reproducibility.

One of the main problems in the micronet analysis consists in the background noise that comes from the solid support. Till now, this problem has been approached through the optimization of the washing conditions, namely the restringency conditions, (steps more or less denaturing, varying the temperature, saline concentration and the presence of detergents) and the attempt to increase the signal quantity and quality that can be obtained from each position on the micronet. In general, it would be ideal to find an alternative way to stabilize the resulting complexes of the specific hybridations in the micronets, in that after the washing step would be increased the number of local/coordinates in the micronets, from which larger signal quantities would be obtained and with a degree of trust, statistically more significant.

On its turn, the development of the biosensor technology has led its application to the most varied fields of activity. In a general way, a biosensor can be defined as an analytical compact device, incorporating a sensitive element of biologic origin. This element is integrated, or intimately associated, to a physical-chemical transducer, capable of producing an electronic signal, proportional to an analyte or group of analytes. The biologic components function in the biosensors therefore is, as in the case of the micronets, to recognize analytes or target molecules. This way, an enzyme will react to the presence of its specific substrate, converting it into a measurable product; antibodies will bind to specific antigens, while the biosensors based on nucleic acids will also depend, as just like the micronets, on the complementarity between the sequence of the probe molecules and the target molecules.

The, most successful commercial biosensor till this date is, without a doubt, the one destined to determine blood glucose content, as it takes on 90% of the market share, quoted in more than 7 billion euros. This instrument is a biosensor example based in the immobilization of an enzyme, glucose oxidase (GOx), on the surface of a transducer electrode coupled to an electrochemical detector. The transducer assembly is done through an electrode enzyme imprinting process, which allows the mass production at low costs.

The commercial success of the biosensor for the blood sugar is due to 2 factors: an increase on the number of patients affected by diabetes and the GOx nature, characterized by a robust structure that allows an immobilization without harmful effects to its activity. The main reason why other biosensors didn't achieve the same commercial success as the blood sugar was due to the instable structural nature of the immobilizing material. Most enzymes, antibodies and nucleic acids are molecules of a frail and mutable nature, especially when removed from their native biologic environment. The means of preservation of the structural and functional integrity of these molecules have become, therefore, vital to the biosensor industry.

### DETAILED DESCRIPTION OF THE INVENTION

This invention is based upon a compatible solute application of biologic materials to immobilize or immobilized, in order to improve the performance of the techniques by using, nucleic acids micronets, and of proteins or biosensors. The compatible solutes in question are selected through a group that includes mannosylglycerate (MG) and its 3 derivates mannosylglyceramide (MGA), mannosylglycerol (MGOH) and mannosyllactate (MGLac), the phosphate glycerol (DGP), ectoin, hydroxyectoin and/or derivates or these compound combinations.

The invention contributes substantially to overcome one of the main problems in the analysis of the micronets, which consists with the background noise which is produce by the solid support. The solution to the problem was to apply the solutes, taking advantage of its stabilizing properties, the storage solutions of the biological material to be immobilized, to the hybridation solution among immobilized probe and target molecules and/or the fixation solution of the probe molecules in the solid supports. The performance improvement of the referred techniques, is shown in this patent through clear examples, that is due to one or several of the following factors: better conservation state of the material to be immobilized, and/or leading to a decrease of the storage requirements and/or transport, a more efficient and uniform deposition of the material on the support, more effectiveness and/or specificity of the marked hybridation material, all this originating an increase of the signal-to-noise-ratio.

Just as the examples that will be described show, the invention is backed-up by clear evidences that the compatible solutes have the ability to preserve biologic material, increasing its integrity in time, not loosing the molecule competence, such as the nucleic acids and proteins, to be experimentally used. That brings great benefits, namely, the long term preservation of the biologic samples, as well as their transport, as the stabilizing ability of the used solutes, work in prolonged temperature intervals, including room temperature, with which the postal transport is less expensive (see examples 1-3).

The compatible solutes were applied as means to stabilize the consequent interactions of specific hybridation from the technologies based on the immobilization of the biologic material, as a way to improve the performance and reliability. For that matter, commercial DNA oligonucleotides networks type GeneChip® from Affymetrix were used, rudimentary protein micronets and biosensors.

The GeneChip® type micronets from Affymetrix, there are multiple pairs of probes (e.g. 11 pairs) for each gene, representing a micronet, chosen from terminal 3′ from the respective mRNA sequence. Each pair consists in a probe which sequence complements perfectly the target sequence (PM—perfect match) and in a probe which sequence, is identical to the PM, with an exception of a single altered base at the centre of the oligonucleotides (MM—mismatch), allowing a quantification and subtraction of the signals from a non-specified hybridation. The sample (target molecules) that is going to be analyzed in the GeneChip® is prepared from cells or tissues, where the total RNA is isolated. This invention shows that the compatible solute application in the micronets hybridation tampon type GeneChip® from Affymatrix, improves its development in what regards the increase of total and real signs. With the consequent increase of the ratio between total signal and background noise, as well as the increase of total numbers or coordinates, in micronets, from which real signals are obtained with a statistically more reliable degree of trust to be used and interpreted through experiment (see example 4).

In the case of protein micronets, the invention was based on a model where, different marked/connected protein epitopes that are connected with glutathione S-transferase (GST), different connection domains linked to the GST and immobilized glass blades covered with poly-l-lisina. In this micronet model, based on immobilized proteins, the comparand pattern was that the same immobilized materials were used, without the presence of the solutes. The different immobilized proteins were detected after hybridation with a primary antibody, followed by a secondary antibody marked with a fluorescent probe. It shown that the application of compatible solutes in an immobilization/fixation solution of the biologic materials in the micronets improves, its performance in what regards efficiency and standardization of the deposit of the material on the support, to the increase of total and real signals, with a consequent ratio increase, as well as the increase of the technical sensibility, allowing to use a smaller amount of immobilized material, in order to obtain quality results (see example 5).

In case of biosensors, these as well as the micronets should show stability on their conservation and storage level, as well as operational stability during its use.

Factors that can destabilize the biosensor, such as, the deactivation of an immobilized enzyme, can be from several different orders, such as, denaturing, the loss of essential co-factors, protein aggregation, irreversible inhibition, proteolysis, microbial degradation to chemical changes to the functional structure. In most cases, the biosensor destabilizing process, involves more than one of these factors. This invention adds a broader solution spectrum, within the strategy that is mostly used, in order to preserve the quality of the immobilized material which will be used in the utilization of the immobilizing solution. Additives such as, neutral polymer or loaded, salts, tampons, compounds destined to modify a hydration layer, such as, low molecular weight sugars (such as, trehalose and sucrose), or poly alcohols (such as, glycerol, sorbitol or lactitol), they have been successfully utilized. Nevertheless, further from the micronet case, the stabilization of the, materials to be immobilized, utilized in biosensors, depends a lot on the specificity of those materials, making it essential to have a broader solution spectrum to clog such diversity. The example 6, described in the same patent show that the compatible solutes can be more effective alternatives to the preservation of the enzymatic protein activity (glucose oxidase) immobilized in biosensors during the storage period.

### EXAMPLE 1

The double chain DNA molecules (dsDNA) can be mismatched/denatured in its two simple complementary chains (ssDNA) increasing the temperature. The temperature which 50% of the base pairs are already mismatched, is called denaturing temperature or melting (Tₘ). It is shown, in this example, that the amount of heat or energy, necessary to denature the double helix DNA, increases the presence of the DGP in the solution. For that reason the clarometry technique was developed, using a model based in two oligonucleotides (S) and S2) complementary to 14 pairs of basis: S1-5′-GCGTCATACAGTGC-3′; S2-5′-GCACTGTATGACGC-3′. The oligonucleotides were dissolved in a plug SSC 0.1×, and dialyzed against the same plug. Its matching was promoted during the incubation at 90° C., during 3 minutes, so that it would eliminate any intermolecular hybridation, followed by a gradual, room temperature, cooling. The double helix thermal denaturing tests were done in a VP-DSC MicroCalorimeter, of MicroCal, between 10° C. and 100° C. and a heat rate of 1° C. per minute. Both calorimeter cells, sample and reference, were subject to pressure of 30 p.s.i. of N₂. All the tests were done at least three times. The DNA concentration used in each test was of 88 uM, having been tested by the DGP and concentration solutions of 0.25 M, 0.5 M and 1 M. The resultant thermo grams were analyzed by the Original program, version 5.0, from MicroCal.

Based on the existing bibliography, the theoretical value of the double helix S1-S2 of its Tm, would be between 52° C. and 53° C., the scores obtained in these tests, have confirmed this information (51.74° C.±0.03° C.). In what regards the DGP action in the stabilizing the double helix, a Tm variation is verified according to the DGP. A maximum stabilization was ascertained with a GDP concentration of 0.3M in solution, from which there are no significant changes in the Tm scores/values. The obtained Tm scores were 68.18° C. (±0.03° C.), 70.14° C. (±0.03° C.) and 70.13° C. (±0.03° C.), for 3 concentrations of DGP tested, 0.25 M, 0.5 M and 1 M (FIG. 1A).

The graphic representation of the thermo grams obtained for the double helix DNA, both with only one plug SSC 0.1% (control), and the three concentrations of DGP (FIG. 1B), it shows well the outcome of the variation of the heat capacity (Cp; Kcal/mole/° C.) of the sample that works according with the temperature. It shows the peak that corresponds to the maximum necessary energy exists, so that it can separate the two DNA chains. This energy corresponds to a given energy, denominated Tm. The observed difference between the control and the sample in the presence of the solute, can be measured through the dislocation of the peaks to the right in the abscissa axis, an indication that the denaturing of the double helix took place at higher temperatures and therefore, was a stabilization of the secondary structure of the DNA molecules, due to the presence of DGP in solution (FIG. 1B).

### EXAMPLE 2

One of the inherent worries in the manipulation of the RNA, as during its application in tests with micronets; is the maintenance of its integrity. It is important to avoid as much as possible, the naturally high rates of RNA degradation, so that an eventual artefact derived from any of the initial sample degradation or that might, during the hybridation process influence the results. That being, the MG, the MGA and the DGP were tested as potential RNA stabilizers in these experiments. Samples of 20 μg of total RNA of Escherichia coli (500 ng/μl) dissolved in water treated for the absence of ribonucleases (treatment with DEPC), which were kept at room temperature (˜23° C.) during 12 weeks, in absence and in the presence of crescent concentrations 50 mM, 100 mM and 250 mM) of MG, MGA and DGP. Samples of 500 ng were harvested during a long period of time and ran by agarose gel electrophoresis in order to verify its integrity. The integrity was measured through the observation of three visible bands, corresponding to three, more abundant RNA ribosomal (rRNA), with sedimentation rates of 30S, 23S and 5S, after the colouring gel, with ethidium bromide, and the incidence with UV radiation. This way, the sRNA kept at room temperature, Without MG, MGA or DGP, started showing signs of degradation by the end of two weeks, whereas, the presence of 50 mM MG, MGA e DGP, delayed the degradation of the in vitro RNA, avoiding its degradation over a period of 12, 6 and 10 weeks respectively (FIG. 2).

### EXAMPLE 3

The stabilizing properties of the compatible solutes MG and DGP that lead to a stabilization of the biologic material, preserving its integrity, during its transport at room temperature (temperature which, the postal transport is less expensive), are shown, in this example, that the total RNA extracted from the cellular line HEK293 (human Embryonic kidney epithelial cells). This way, the total RNA, was extracted according to the NucleoSpin RNAII-Kit (Macherey-Nagel) protocol. The samples (a sample containing only total RNA in H₂O-DEPC and two samples containing the total RNA in H₂O-DEPC and 25 mM MG or 25 mM DGP), were sent through the post and subject to room temperature and according to its variations while being transported during a period of 96 hours, from the university of Ferrrara, Ferrara Italy, to the STAB VIDA Lda. laboratory in Oeiras, Portugal. As means of control, a sample of the same RNA was used and kept at −80° C., during the transport period (resorting to dry ice). In order to analyze the integrity of these samples, the capillary electrophoresis, a bio-analyser of the Agilent brand was used. The results of the analysis by capillary electrophoresis, shows that the samples that contain solutes, as well as the sample preserved at −80° C. are wholly preserved, showing evidences of a presence of 3 bands and 3 peaks in the gel and in the fluorograms respectively, corresponding to the 3 more abundant shapes of ribosomal RNA (rRNA) with sedimentation rates of 30S, 23S and 5S respectively. On its turn, the sample carried in the absence of compatible solutes, which was totally degraded (it is verified due to the presence of several bands, corresponding to the degradation products, in the gel photograph, and of the several peaks in the fluorogram—FIG. 3A).

Competence studies were done in order to verify if the presence of compatible solutes in functionary solution, as an inhibitor or inducer of reactions, such as, the complementary DNA synthesis (cDNA) from total RNA, which would use this preserved RNA as a mould. For that matter several commercial kits for the cDNA were used: cloned AMV, Thermo script and Superscript III, all from Invitrogen. According to the results, it is verified that both MG and DGP, apart from preserving the total RNA integrity in transport conditions; they would also not interfere with the biologic performance of the RNA, having this RNA, competently used as a mould for the cDNA synthesis (the presence of cDNA was visualized as agarose gel after the electrophoresis, after dragging the correspondent cDNA, produce from the mRNA different dimensions—FIG. 3B). It was indeed possible to verify that with the utilization of the 3 kits occurred consistently to the formation of the cDNA, from the samples preserved in the presence of MG or DGP, as from the control sample, preserved at −80° C.

### EXAMPLE 4

In this example it is demonstrated how the application of compatible solutes in a DNA hybridation micronets plug, more specifically, the GeneChip® micronet type from Affymetrix; improves their performance in what regards the increase of total and real signals, with a consequent increase of the ratio between total signal and background noise, as well as of the number of locals/coordinates in the micronets from which the real signals are obtained, with a degree of trust, statistically more reliable in order to be, used and experimentally interpreted. The protocol used went according to the standard advised by Affymetrix company http://www.affymetrix.com/support/technical/manual/expression_manual.affx). Thus the total RNA was extracted according to the NucleoSpin RNAII-Kit protocol (Macherey-Nagel), from the cellular line HEK293 (human Embryonic kidney epithelial cells). This RNA was afterwards used to; synthesize the double chain cDNA, containing a sequence of the T7 promoter in its terminal 5′. This region was therefore used for in vitro transcription, in which the nucleotides marked with biotin (biotin-ddUTP and biotin-ddCTP), were incorporated, obtaining therefore the RNAc. This second step allowed an approximate increase of 100× of the initial material allowing, the use of only 5 μg of the total initial RNA. The marked RNA was then fragmented, which outcome were molecules with sizes that vary between 35-200 bases, hybridized in the GeneChip® for 16 hours after marking with a conjugate of fluorescent estreptavidine. After washing in an Affymetrix fluidic station, the intensity of the fluorescence in each micronet coordinate was measured with an Affymetrix GeneChip Scanner 3000 detector, at a wavelength of 570 nm. The GeneChip® type micronets used were specifically GeneChip® Test Arrays. These micronets contain groups which contain 345 target molecules, consisting in oligonucleotides, with sizes that vary between 16 to 25 nucleotide lengths, representing highly preserved DNA sequences among species.

The experimental plan consisted in the trebled hybridation of (3 samples) of GeneChip® Test Arrays, allowing the composition hybridation solution to vary solely, in what regards the content in terms of compatible solutes. Each one of the solutes was added separately to 3 different concentrations (50 mM, 150 mM and 300 mM), in which the control consisted in a trebled (a parallel for each concentration), of tests in which, no additive was added to the hybridation solution. Three levels of analysis results were carried out:
1. The average intensity of the real signal obtained in the presence of solutes in its totality vs. average real signal intensity obtained in the absence of solutes.
2. Average real signal intensity obtained in the presence of each one of the solutes, individually vs. average real signal intensity obtained in the absence of solutes.
3. Average real signal intensity of each sample vs. real signal intensity obtained from the hybridized control in parallel (in the same series).

The results of the analysis are summarized in the following tables:

Table I level 1 of the analysis results of the results: Average intensity of the real signal obtained in the presence of solutes in its totality vs. average real signal intensity obtained in the absence of solutes (controls).

TABLE 1

| | Average of | |
|---|---|---|
| | controls | Average with solutes |
| Total Intensity (with | 189260.6 | 173308.6 |
| background noise) | | |
| Real intensity (without | 117704.9 | 121177.3 |
| noise)* | | |
| % Real intensity of the | | +3.0% |
| Samples | | |
| vs Real intensity of | | |
| controls | | |
| Intensity of the background | 71555.7 | 52131.4 |
| noise* | | |
| % Intensity of noise of the | | −27.1% |
| samples | | |
| vs Intensity of the noise of | | |
| the controls | | |
| Total intensity/background | 2.64 | 3.32 |
| noise | | |
| % Increase of total intensity/ | | +25.7% |
| Increase of background | | |
| noise | | |

*p < 0.001

In its totality and according to table I, the presence of compatible solutes in the hybridation solution increases the intensity of the real signal in an average of 3%, reduces the background noise in an average of 27%, and increases the ratio between the average total signal and the background noise in 25.7%.

Table II Level 2 of the analysis of the results: the average intensity of the real signal obtained in the presence of each one of the solutes, individually vs. the average real signal obtained in the absence of solutes.

TABLE 2

| | | Average in | Average in | Average in | Average in | Average in |
|---|---|---|---|---|---|---|
| | | the | the | the | the | the |
| | Average of | presence | presence | presence | presence | presence |
| | controls | of DGP | of ECT | of HECT | of MG | of MGLac |
| Total | 189260.6 | 155205.4 | 188206.8 | 183128.9 | 153973.9 | 186028.3 |
| intensity | | | | | | |
| (with | | | | | | |
| background | | | | | | |
| noise) | | | | | | |
| Real | 117704.9 | 116209.4 | 120918.1 | 139132.9 | 111499.2 | 118126.8 |
| intensity | | | | | | |
| (without | | | | | | |
| noise) * | | | | | | |
| % Real | | −1.3% | +2.7% | +18.2% | −5.3% | +0.4% |
| intensity of | | | | | | |
| samples | | | | | | |
| vs Real | | | | | | |
| Intensity of | | | | | | |
| controls | | | | | | |
| Intensity of | 71555.7 | 38996.0 | 67288.7 | 43996.0 | 42474.7 | 67901.5 |
| Background | | | | | | |
| noise ** | | | | | | |
| % Intensity | | −45.6% | −6.0% | −38.5% | −40.7% | −5.1% |
| of the noise | | | | | | |
| of the samples | | | | | | |
| vs Intensity | | | | | | |
| of the noise | | | | | | |
| of the controls | | | | | | |
| Total | 2.64 | 3.98 | 2.80 | 4.16 | 3.63 | 2.74 |
| intensity/ | | | | | | |
| background | | | | | | |
| noise | | | | | | |
| % Increase | | +50.5% | +5.8% | +57.4% | +37.1% | +3.6% |
| of total | | | | | | |
| intensity/ | | | | | | |
| Increase of | | | | | | |
| background | | | | | | |
| noise | | | | | | |

p < 0.05
** p < 0.001

According to table II, the HECT caused a significant induction, 18.2% of the real signal; all the compatible solutes caused individually, a background noise reduction in regards to the controls, with special incidence in DGP, HECT and MG. As a result, all the solutes, individually, caused an increase in the ratio between the intensity of the real signal and the background noise, with special incidence in the three previously referred compounds.

Table III Level 3 of the analysis of the results: the intensity of the real signal of each sample vs. the intensity of the real signal obtained from the hybridized control in parallel (same series).

TABLE 3

| | Ratio of values p:p (control)/p (sample) | | |
|---|---|---|---|
| | Where p is the number of locals, or | | |
| | coordinates, in the micronets considered | | |
| | by the AffyMetrix GOCS software as being | | |
| | emission locals to considerer | | |
| | Control | Control | Control |
| | series 1 | series 2 | series 3 |
| 1. DGP 150 mM | 0.014 | | |
| 2. DGP 300 mM | | 0.487 | |
| 3. DGP 50 mM | | | 0.262 |
| 1. ECT 150 mM | 0.044 | | |
| 2. ECT 300 mM | | 0.058 | |
| 3. ECT 50 mM | | | 0.875 |
| 1. HECT 150 mM | 0.05 | | |
| 2. HECT 300 mM | | 0.502 | |
| 3. HECT 50 mM | | | 0.506 |
| 1. MG 150 mM | 0.008 | | |
| 2. MG 300 mM | | 0.735 | |
| 3. MG 50 mM | | | 0.431 |
| 1. MGLac 150 mM | 0.161 | | |
| 2. MGLac 300 mM | | 0.585 | |
| 3. MGLac 50 mM | | | 0.615 |

According with the table III, the concentration which, the compatible solutes induced the total signal in order to increase the number of locals, or coordinates, in the micronet considered by the software AffyMetrix GOCS, as being locals of emission to considerer, was of 150 nM. To this concentration: the DGP, the ECT, the HECT and the MG, ratios p present (control)/p (sample)≦0.05 related to the total signal, representing a dramatic increase of the technical sensibility.

### EXAMPLE 5

In this example, it is shown how the application of compatible solutes in the immobilization/fixation solution of the biologic materials in the protein micronets, improves their performance, in what regards the increase between total signal and the background noise.

For that matter, 16 micronets consisting of glassless blade poly-L-lisined were used.

In these micronets, were imprinted and immobilized 4 different epitopes of the protein PIN1 (100 m g/ml): PIN1, PIN2, C109A, T23A. The immobilization/fixation solution consisted of PBS, containing each one of the solutes at a final concentration of 150 mM or, how controls, glycerol 150 mM and glycerol 20% (2.7M—already commonly used). Were also imprinted in locals/coordinates in the micronets, only solutes, in order to, have a negative control, in regards to, the non-specified hybridation with antibodies used as target molecules. The imprint was done with the help of a robotized imprinter BioRobotics MicroGrid II, at 30° C. and 60% of humanity. The micronets were dried and stored at 4° C. until its utilization. The PIN1 immobilized protein was detected by direct hybridation with a primary antibody anti-PIN1 rabbit polyclonal and afterwards with a secondary antibody mixture, anti rabbit, marked with fluorophor cianine-3 (Cy3), bright pink and cianine-5 (Cy5), blue.

All the procedures regarding hybridation and the wash of the micronets were done at room temperature. Before the hybridation, the micronets were rinsed with PBS 1×, for 2 minutes and blocked with dissolved powdered milk in PBS1× ((3% p/v) for 2 hours. Afterwards were washed 3 times with PBS 1× (5 minutes each wash) and proceeded to the hybridation with a primary antibody [powdered milk 5% (p/v), primary antibody (1:1000), 0.1% (v/v) and NaN₃ 0.02% (p/v) in PBS 1×]. It was washed again 3 times with PBS 1× (5 minutes each wash) proceeding afterwards to the hybridation with secondary antibodies mixture marked [powdered milk 5% (p/v), secondary antibody bright pink fluorophor (100 m g/ml) 0.4% (v/v), secondary antibody with blue fluorophor (100 m g/ml) 0.4% (v/v) and NaN₃ 0.02% (p/v) in PBS 1×]. From this step, the micronets were covered with foil to avoid photodecomposition. It proceeded to a new wash—3 times with PBS 1× (5 minutes each) and a last incubation with distilled water. The drying was done by centrifugation during 5 minutes, a 20° C. and a 1700 rpm.

Finally, the hybridized micronets were submitted to a laser emission detector radiation beam (Affymetrix 428™); the images obtained were analyzed and the results processed with the help of the BioDiscovery, Inc ImaGene™ 4.0 programme and the programme of Microsoft Excel. During the analysis, the signal intensities were normalized using the Global Median method.

Table IV The table shows the real signal for two channels Cy5 and Cy3, which consists in the average median of the signal (corrected for the background noise) proceeding from the 4 epitopes different from the PIN1 protein, measured from 16 independently hybridized micronets in absence (control) and in the presence of several additives in the immobilization/fixation solution. Also shows a change of the real signal in what regards the control to the glycerol 20% (commonly used).

TABLE 4

| | Real Signal | | Change of the real | | Change of the real | |
|---|---|---|---|---|---|---|
| | (without back- | | signal regarding | | signal regarding- | |
| | ground noise) | | the control | | glycerol 20% | |
| Solute | Cy5 | Cy3 | Cy5 | Cy3 | Cy5 | Cy3 |
| Glycerol | 2267.02 | 3174.76 | 161% | 120% | — | — |
| 20% | | | | | | |
| Glycerol | 515.66 | 1096.96 | −30% | −24% | −73% | −65% |
| DGP | 3933.26 | 7570.34 | 504% | 425% | 132% | 138% |
| MGLac | 681.87 | 1258.38 | −10% | −13% | −65% | −60% |
| MGA | 2436.55 | 2955.46 | 150% | 105% | −4% | −7% |
| MG | 1879.59 | 3827.14 | 226% | 166% | 25% | 21% |
| HECT | 2107.48 | 3425.98 | 186% | 138% | 10% | 8% |
| ECT | 809.42 | 1234.00 | −3% | −14% | −63% | −61% |
| Control | 642.99 | 1441.31 | — | — | −62% | −55% |

From the analysis envisaging the comparative determination of the real signal, in absence (control) and presence of compatible solutes, summarized in table IV, it is verified that, both in the presence of DGP, MG or HECT, that there is significant increase of the real signal obtained from the micronets. This increase regarding the real signal is a lot more significant in regards the control, in the absence of any additive (FIG. 4A), but it maintains, in what regards the glycerol 20% (commonly used), showing that the application of compatible solutes (namely DGP) are more effective than the methods used, now-a-days for that same purpose.

Table V The table shows a ratio between total signal and the background noise for both channels, which consist in the average medians of the signal and the background noise respectively, proceeding from the 4 epitopes different, from the PIN1 protein, measured from the micronets independently hybridized in absence (absence) and in the presence of various additives in the mobilization/fixation solution. Also shows a change of this ratio in what regards the control to the glycerol 20% (commonly used).

TABLE 5

| | Total signal/ | | | | | |
|---|---|---|---|---|---|---|
| | Background noise | | S/R additive vs | | S/R additive vs | |
| | (S/R) | | Control | | Glycerol 20% | |
| Solute | Cy5 | Cy3 | Cy5 | Cy3 | Cy5 | Cy3 |
| Glycerol | 1.60 | 1.40 | 147% | 90% | | |
| 20% | | | | | | |
| Glycerol | 0.48 | 0.71 | −26% | −4% | −70% | −50% |
| DGP | 2.68 | 2.50 | 312% | 240% | 67% | 79% |
| MGLac | 0.60 | 0.70 | −7% | −5% | −62% | −50% |
| MGA | 0.65 | 1.62 | 178% | 121% | 13% | −47% |
| MG | 1.81 | 2.36 | 319% | 221% | 70% | 16% |
| HECT | 2.72 | 1.74 | 202% | 136% | 23% | 69% |
| ECT | 1.97 | 1.69 | 187% | 130% | 16% | 24% |
| Control | 1.87 | 0.73 | | | −59% | 21% |

From the analysis of the results aiming at the comparative determination of the ratios between total signal and the background noise, in absence (control) and presence of compatible solutes, summarized in table V, it is verified that, both in the presence of DGP, MG, HECT, there is a significant increase of this ratio. This relative ratio increase between the total signal and the background noise is a lot more significant in what regards the control, in the absence of any additive (see FIG. 4B), but it maintains in regards to glycerol 20% (commonly used), demonstrating that the application of compatible solutes (namely the DGP) is more effective than the methods used now-a-days, for the same purpose. The increase of the ratio between the total signal and the background noise has direct repercussions in the sensitivity and reproducibility of the technique.

### EXAMPLE 6

In this example it is shown how the application of compatible solutes of enzyme immobilization/fixation (glucose oxidase—GOx) in biosensors improves their performance in what regards the increase of the enzyme activity after a period of storage.

The model biosensor used in this example is based in glucose oxidation, on behalf of GOx, being the resulting electrons of this oxidation transferred through the prosthetic group of the enzyme, for the di-oxygen present in the middle of the sample. The di-oxygen is capable of functioning as an electron acceptor, being therefore reduced to hydrogen peroxide. The hydrogen peroxide is then amperometrically detected, through an electrode fixed to a determinate potential which facilitates the oxidation reaction, with subsequent transfer of electrons to the active electrode.

In this example the electrochemical transducer takes the shape of a net imprinted according to the method described in Kroger et al. (1998). Consists in; an active rodomised carbon electrode, a counter carbon electrode and a reference electrode in silver/silver chloride (Ag/AgCl₂). The rodomised carbon electrode promotes electrocatalysis reducing the potential to which the hydrogen peroxide is oxidated.

In order to prepare the electrodes aliquotas of GOx (volumes of 10 ml containing 2U ml⁻¹ in plug Na₂PO₄ 0.1 M, pH 7.2) are pipetted inside of their own structures in the active electrode and left to dry at room temperature (˜23° C., 16 h). The rodomised carbon matrix contains 2.5% p/v of a polymer of hydroxi-etil-celulose, soluble in water, which was, previously stabilized for the utilization in an aqueous mean through a heat treatment (120° C., 2 h). This matrix has proven already to be quite effective for the immobilization of solutions containing proteins due to its hydrophilic nature and the physical-chemical properties of the carbon matrix. The biosensors are finally stored at 4° C., in vacuum conditions, until its utilization.

In the case of this example, the electrodes were prepared as described above, with the difference that, in cases where the compatible solutes were applied, 10 m l of selected compound were applied, immediately after the addition of GOx, followed by a homogenization of the mixture through the soft agitation, in order to avoid damaging the surface of the active electrode. The optimum concentrations for each one of the compounds were previously determined as being: ECT (100 mM); HECT (80 mM); MGA (50 mM); DGP (100 mM) and MGLac (70 mM). The biosensors were then tested after 8 months of conservation. Volumes of 25 m l de glucose 20 mM were applied in a phosphate plug, pH 7.2/0.1 M KCl. The active electrode was then subject to a potential of +350 mV (against the electrode of reference Ag/AgCl₂) and the current in the device, measured after 150 seconds.

According with the results (FIG. 5) there was an increase in the retention of the activity of the GOx (current>0.5 m A) in presence of all the compatible solutes, tested in what regards the electrodes control without stabilizers (current<0.4 m A). The improvements in the preservation of the GOx activity were evident with the addition of the DGP, MGA and HECT.

### DESCRIPTION OF THE FIGURES

FIG. 1 Action of the DGP in the stabilization of the double helix duplex S1/S2. A. The graphic shows an increase of the Tm of the duplex S1/S2 in function of the concentration of the DGP, measured through the technique of calorimetry. A maximum stabilization is reached with DGP at a concentration of 0.3 M. The Tm value obtained were respectively 68.18° C. (±0.03° C.), 70.14° C. (±0.03° C.) and 70.13° C. (±0.03° C.), for the three DGP tested concentrations, 0.25 M, 0.5 M and 1 M. B. Graphic representation of the thermo grams obtained for the S1/S2 DNA duplex double helix, both in the presence of just the plug SSC 0.1 (control), as well as the three DGP concentrations. The graphic shows a variation of the heat capacity (Cp; Kcal/mole/° C.) of the sample in function of the temperature. It is verified that there is a peak that corresponds to the maximum necessary energy for the separation of the DNA chain. The difference observed between the control and the sample in the presence of the solute, can be measured through the dislocation of the peaks to the right abscissa axis, a sign that a double helix denaturation has taken place at higher temperatures.

FIG. 2 Stabilization of the total RNA to room temperature. Sample of 20 m g of RNA (500 ng/m l) extracted from Escherichia coli were dissolved in water treated for the absence of ribonucleases (treatment with DEPC) and maintained at room temperature (˜24° C.) during 12 weeks in absence and the presence of crescent/increasing concentrations (50 mM, 100 mM and 250 mM) of MG, MGA and DGP. Samples of 500 ng were collected during a long period of time and run through the agarose gel in electrophorese in order to verify its integrity. The integrity was measured through the observation of three visible bands, corresponding to the three more abundant shapes of ribosomal RNA (RNAc), with sedimentation levels 30S, 23S and 5S respectively, after the colouring of the gel with ethidium bromide, and incidence with UV radiation. This way, the RNAs maintained at room temperature without MG, MGA or DGP started showing signs of degradation at the end of two weeks, while the presence of 50 mM MG, MGA and DGP, delayed the in vitro RNA degradation, avoiding its degradation during period of 12, 6 and 10 weeks respectively. The molecular weight marker, run in parallel with samples was the Fago DNA 1 digested with PstI.

FIG. 3 RNA stabilization on behalf of the MG and DGP preserving its integrity and functionality during the transport. A In order to analyze the integrity of the RNA, it was appealed to the capillary electrophorese using a Bio-analyzer of the brand Agilent. The figure shows the results of the capillary electrophorese analysis where it is verified that the samples containing the solutes, as well as the sample preserved at −80° C., presented themselves whole, evidencing themselves if the presence of 3 bands and of 3 peaks in gel and in the fluorograms respectively, corresponding to the 3 more abundant shapes of ribossomal RNA (RNAr) with levels of sedimentation 30S, 23S and 5S respectively. On its turn, the transported sample in the absence of compatible solutes was totally degraded (that is verified by the presence of several bands corresponding to the degradation products), in the gel photography, and several peaks in the fluorogram. B. In order to analyze the biologic functionality, several commercial kits for the cDNA synthesis (cloned AMV, thermoscript and yet superscript III, all proceeding from introgen) from the RNA preserved in the conditions described above. The presence of cDNA was visualized in agarose gel in electrophorese through the dragging, which corresponds to cDNA from the RNAc with different dimensions. Its was possible to verify that with the utilization of three kits, consistently occurred the formation of cDNA from the samples preserved in the presence of MG or DGP, as well as from the control sample, preserved at −80° C.

FIG. 4 Relative increase of the real signal and the ratio between the total signal and the background noise in the obtained results from the protein micronets, derived from the compatible solute addition to the immobilization/fixation solution. A. The graphic shows a variation of the real signal, in what regards the control, for two channels, Cy5 and Cy3, depending on the additive. The signal was determined from the average medians of the signal (corrected for the background noise) proceeding from 4 different PIN1 protein epitopes measured from the 16 independently hybridized micronets.

FIG. 5 Influence of compatible solutes in the glucose oxidase (GOx) activity, immobilized in biosensors during the storage period. Biosensors with active electrodes based on immobilized GOx were stored for 8 months at 4° C. The GOx activity was measured through the current generated by the glucose oxidation. The control refers to electrodes prepared without the presence of any compatible solute. Concentrations of solutes in immobilization/fixation solution of GOx were: ECT 100 mM; HECT 80 mM; MGA 50 mM; DGP 100 mM and MGLac 70 mM.

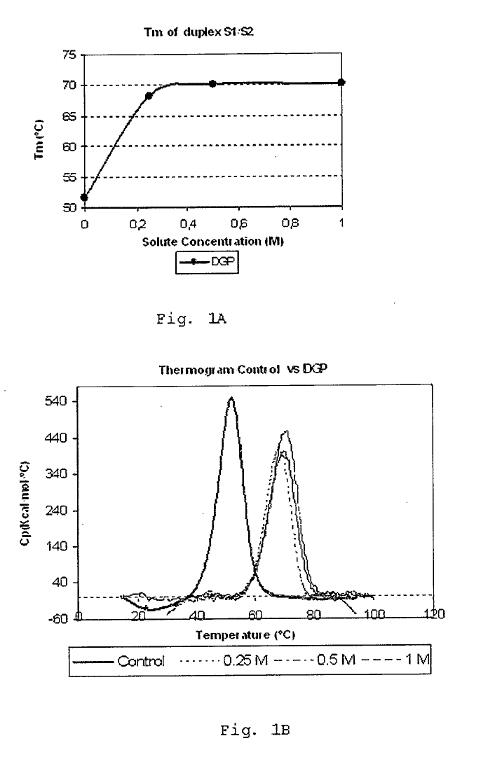

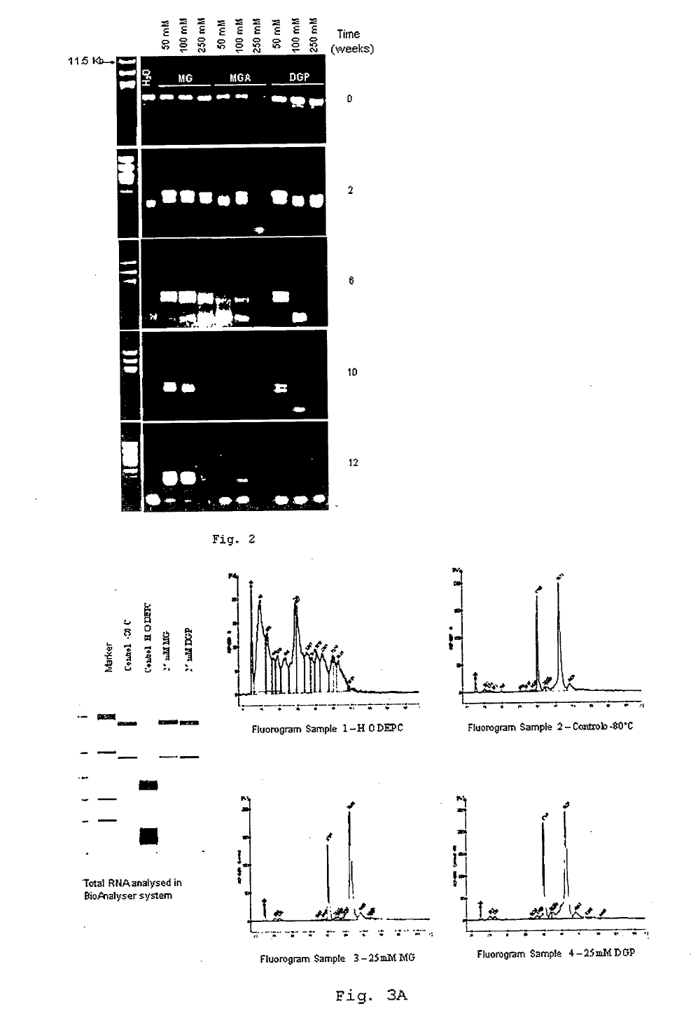

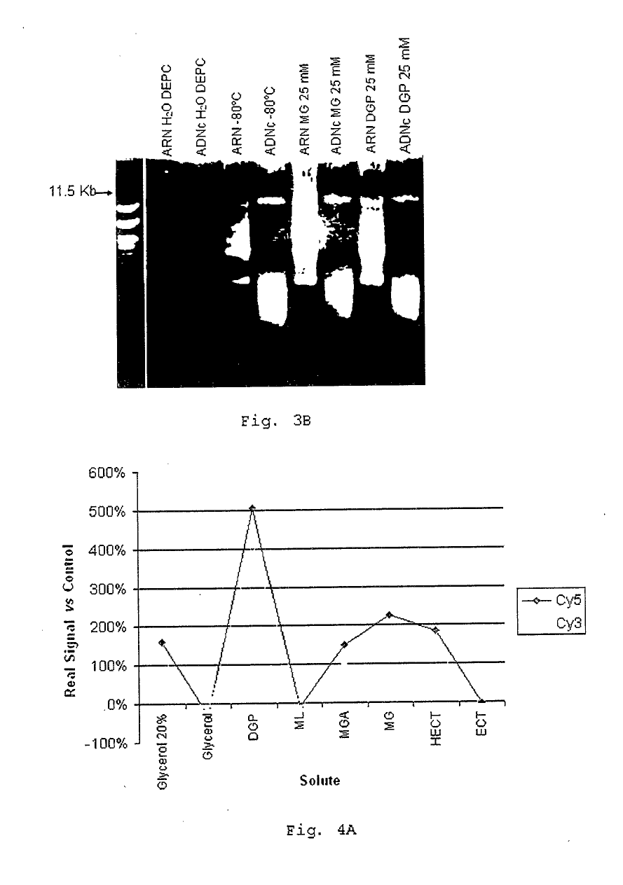

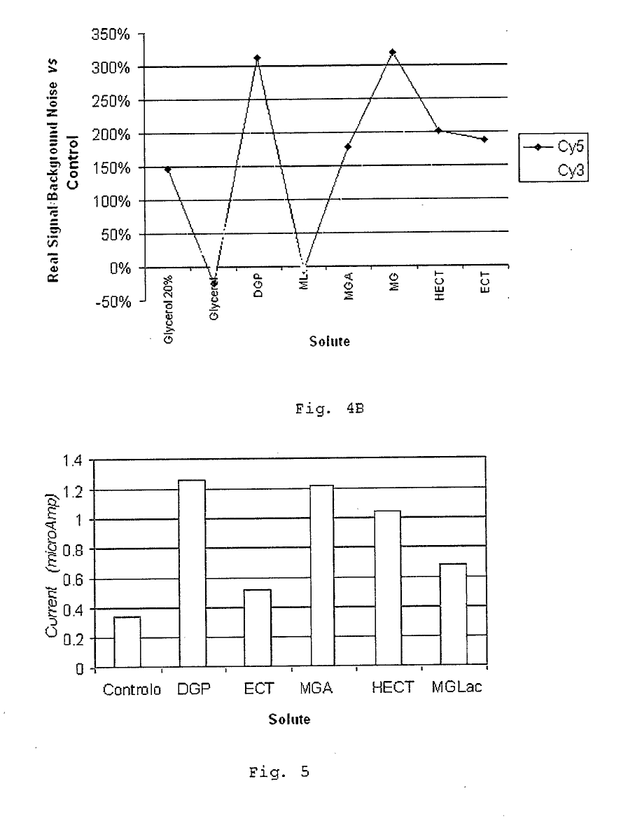

### LITERARY REFERENCES

Arakawa, T., and Timasheff, S. N. 1985. J. 47:411-414.
Bouveng, H., Lindberg, B., and Wickberg, B. 1955. Acta Chem. Scand. 9:807-809.
Breslauer, K. J., Frank, R., Blocker, H., and Marky, L. A. 1986. Proc. Natl. Acad. Sci. USA. 83 :3746-3750.

Brown, A. D. 1976 Microbial water stress. Bacteriol. Rev. 40:803-846.
Frackman, S.; Kobs, G., Simpson, D. and Storts, D., Betaine and DMSO, enhancing agents for PCR, Promega notes, http://www.promega.com/pnotes
Freier, S. M., Kierzek, R., Jaeger, J. A., Sugimoto, N., Caruthers, M. H., Neilson, T. and Turner, D. H. 1986. Proc. Natl. Acad. Sci. USA. 83:9373-9377.
Knapp, S., Ladenstein, R., and Galinski, E. A. 1999. Extremophiles 3:191-198.
Kremer, B. P., and R. Vogl. 1975. Phytochemistry 14:1309-1314.
Kroger, S., S. J. Setford, and A. P. F. Turner. 1998. Anal. Chem. 70:5047-5053.
Lamosa, P., L. O. Martins, M. S. da Costa, and H. Santos. 1998. Appl. Environ. Microbiol. 64:3591-3598.
Lamosa, P., Burke, A., Peist, R., Huber, R., Liu, M. Y., Silva, G., Rodrigues-Pousada, C., LeGall, J., Maycock, C., and Santos, H. 2000. Microbiol. 66: 1974-1979.
Le Bret, M. and Zimm, B. H. 1984 Biopolymers 23:287-312.
Malin, G., Iakobashvili, R., and Lapidot, A. 1999. Type II restriction endonucleases as a model system. J. Biol. Chem. 274:6920-6929.
Manning, G. S. Q. 1978. Rev. Biophys. 11:179-246.
Martins, L. O., and H. Santos. 1995. Appl. Environ. Microbiol. 61:3299-3303.
Nunes, O. C., C. M. Manaia, M. S. da Costa, and H. Santos. 1995. Appl. Environ. Microbiol. 61:2351-2357.
Ramos, A., N. D. H. Raven, R. J. Sharp, S. Bartolucci, M. Rossi, R. Cannio, J. Lebbink, J. Van der Oost, W. M. De Vos, and H. Santos. 1997. Appl. Environ. Microbiol. 63:4020-4025.
Record, M. T, Anderson, C. F., and Lohman, T. M. 1978. Q. Rev. Biophys. 11:103-178.
Rees, W. A., Yager, T. D., Korte, J. and von Hippel, P. H. 1993. Biochem., 32:137-144.
Santos, H., and M. S. da Costa. 2002. Environ Microbiol. 4:501-509.
Santos, H., A. Ramos, and M. S. da Costa. 1998. Thermostabilization, osmo-protection, and protection against desiccation of enzymes, cell components and cells by manosilglicerato. European Patent no. EP0816509.
Schwartz, T. 2003. Use of compatible solutes as substances having free radical scavenging properties. United States Patent Application Publication.

1. Improvement of the performance of utilization for commercial and academic purposes of the biological materials, such as, tissues, cells, proteins, enzymes, and nucleic acids, through the use of compatible solutes as adjuvants in preservation, transport, preparation and use of the same biological materials, to immobilize or immobilized on support, namely, in micronets and biosensors, leading to an increase of:
i) Its net time utilization,
ii) signal-to-noise-ratio or sensitivity according to the techniques used,
iii) Reproducibility in the output of the same techniques.

2. Utilization of compatible solutes according to claim 1, wherein di-glycerol phosphate is used as a compatible adjuvant solute.

3. Utilization of compatible solutes according to claim 1, wherein mannosylglycerate is used as a compatible adjuvant solute.

4. Utilization of compatible solutes according to claim 1, wherein mannosylglyceramide is used as a compatible adjuvant solute.

5. Utilization of compatible solutes according to claim 1, wherein mannosylglycerol is used as a compatible adjuvant solute.

6. Utilization of compatible solutes according to claim 1, wherein mannosyllactate is used as a compatible adjuvant solute.

7. Utilization of compatible solutes according to claim 1, wherein ectoin is used as a compatible adjuvant solute.

8. Utilization of compatible solutes according to claim 1, wherein hydroxyectoin is used as a compatible adjuvant solute.

9. Utilization of compatible solutes according to claim 1, wherein derivatives of di-glycerol phosphate are used or combinations thereof as adjuvants to the performance of techniques using immobilized biological materials.

10. Utilization of compatible solutes in biological materials according to claim 1, wherein the mixture in solution and there is an increase in the net time utilization, in signal-to-noise ratio or sensitivity, and in the reproducibility of the output when these materials are immobilized on DNA micronets.

11. Utilization of compatible solutes in biologic materials according to claim 1, wherein the mixture in solution and there is an increase in the net time utilization, in signal-to-noise ratio or sensitivity, and the reproducibility of the output when these materials are immobilized on cDNA micronets.

12. Utilization of compatible solutes in biologic materials according to claim 1, wherein the mixture in solution and there is an increase in the net time utilization, in signal-to-noise ratio or sensitivity, and the reproducibility of the output when these materials are immobilized in RNA micronets.

13. Utilization of compatible solutes in biologic materials according to claim 1, wherein the mixture in solution and there is an increase in the net time utilization, in signal-to-noise ratio or sensitivity, and the reproducibility of the output when these materials are immobilized on protein micronets.

14. Utilization of compatible solutes in biologic materials according to claim 1, wherein the mixture in solution and there is an increase in the net time utilization, in signal-to-noise ratio or sensitivity, and the reproducibility of the output when these materials are immobilized on biosensors, in this context the term biosensor regarding its official definition by the IUPAC.

15. Utilization of compatible solutes according to claim 1, wherein the mixture in solution and there is an elongation of over 100% of the operating life of the immobilized biologic material.

16. Utilization of compatible solutes according to claim 1, wherein the mixture in solution and protection against degradation and loss of activity of the immobilized biologic materials due to transport are induced.

17. Utilization of compatible solutes according to claim 1, wherein the mixture in solution and a protection against degradation and loss of activity of the immobilized biologic materials due to the storage time are induced.

18. Utilization of compatible solutes according to claim 1, wherein the mixture in solution and an increase of the signal-to-noise ratio or sensitization of techniques using immobilized biological materials, other than those mentioned in claim 1 is induced.

19. Utilization of compatible solutes on the protection of biologic materials according to claim 1, wherein the mixture in solution and is induced an increase of the signal-to-noise ratio or sensitization of techniques using immobilized biological materials, due to a higher efficiency on the immobilization of the mentioned materials, being such increase due to higher quantity of immobilized material, a better quality of the immobilized material or a more uniform deposition of the same material.

20. Utilization of compatible solutes in biologic materials protection according to claim 1, wherein the mixture in solution and is induced an increase of the signal-to-noise ratio or the sensitization of techniques using immobilized biological materials being due to a decrease of the background noise, being such decrease due to a higher specific hybridation, or to a higher specificity of the washing steps.

21. Utilization of compatible solutes on the protection of biologic materials according to claim 1, wherein an increase on the reproducibility of the output of the techniques is originated, using immobilized biologic materials.

22. Utilization of compatible solutes on the protection of biologic materials according to claim, wherein a better conservation state of the biologic material to be immobilized is originated, namely, the in vitro nucleic acids.

23. Utilization of compatible solutes on the protection of biologic materials according to claim 1, wherein the mixture in solution and a better conservation state of the biologic material to immobilize, hybridize and/or manipulate, is induced, being due to a thermal or structural stabilization of the in vitro nucleic acids.

24. Utilization of compatible solutes on the protection of biologic materials according to claim 1, wherein the mixture in solution and a better conservation state of the biologic material to immobilize, hybridize and/or manipulate, is induced, being due to an antidegradation, enzymatic or oxidative action of compatible solutes on in vitro nucleic acids.
